(19) United States
(12) Patent Application Publication
(10) Pub. No.: US 2025/0246313 A1
(43) Pub. Date: Jul. 31, 2025

(54) AUTOMATED RISK ASSESSMENT FOR DEEP VEIN THROMBOSIS AND PULMONARY EMBOLISM USING RETINAL IMAGES

(71) Applicant: Welch Allyn, Inc., Skaneateles Falls, NY (US)

(72) Inventors: Stacie Lynn Brough, Syracuse, NY (US); Hongying Krause, Cranleigh (GB)

(21) Appl. No.: 19/038,915

(22) Filed: Jan. 28, 2025

Related U.S. Application Data
(60) Provisional application No. 63/626,229, filed on Jan. 29, 2024.

(51) Int. Cl. G16H 50/30; A61B 3/14; G16H 10/60; G16H 30/40; G16H 50/20

(52) U.S. Cl. CPC G16H 50/30; A61B 3/14; G16H 10/60; G16H 30/40; G16H 50/20

(57) ABSTRACT

A patient screening system for providing recommendations for screening of a hospitalized patient for risk of developing deep vein thrombosis (DVT) or pulmonary embolism (PE) based on their health records and retinal images, is described herein. The patient screening system may include an optical imaging device for capturing retinal images, and a DVT risk assessment system configured to generate the recommendation for further screening tests. The DVT risk assessment system may implement various AI/ML models trained on a training dataset of anonymized patient data. The patient screening system may also implement detectors for various ophthalmic features correlated with blood clot-related conditions of the patient. Any patient screening based on the recommendation may be followed up, and results of such screening used to improve performance of the patient screening system.

### RELATED APPLICATIONS

This patent application is a nonprovisional of and claims priority to U.S. Provisional Patent Application No. 63/626,229, entitled “AUTOMATED RISK ASSESSMENT FOR DEEP VEIN THROMBOSIS AND PULMONARY EMBOLISM USING RETINAL IMAGES,” filed on Jan. 29, 2024, the entirety of which is incorporated herein by reference.

### TECHNICAL FIELD

This application relates to techniques for automatically determining risk of deep vein thrombosis and pulmonary embolism in hospitalized patients based on retinal images and patient health records, and providing recommendations for further screening related to the risk of occurrence of these conditions.

### BACKGROUND

Pulmonary embolism (PE) or blood clots in the lungs are one of the leading causes of preventable hospital deaths. PE may start with a deep vein thrombosis (DVT) condition where a blood clot forms in a vein. Hospitalized patients present many of the major risk factors for DVT such as physical trauma, surgery, and prolonged immobility from injury or illness. Additionally, underlying conditions that increase risk of DVT are also present in a large percentage of the patients e.g., diabetes (10.5% of the US population), using hormonal contraception (21% of US female population age 15-49), being overweight (30.7% of US adults) or obese (42.4% of US adults), and recent surgery (11.4 million procedures performed annually in US hospitals). As a result, there is a need to regularly screen a hospitalized patient for risk of formation of blood clots so that a DVT condition is detected and treated early in order to avoid a potentially fatal condition (e.g., before blood clots reach the lungs, brain, or heart).

Retinal scans may be helpful in detecting these conditions noninvasively and painlessly in patients. For example, retinal artery or retinal vein occlusion (RVO), which is a blockage or blood clot in one of the small arteries or veins that carry blood to and from the retina in a patient's eye, may be an indicator of clots elsewhere in the body. However, retinal scans are currently not included in the standard of care in hospitals, and a hospitalized patient is not assessed for risk of developing blood clots based on their retinal scans, in conjunction with the patient's medical records.

The various examples of the present disclosure are directed toward overcoming one or more of the deficiencies noted above.

### SUMMARY

In various examples of the present disclosure, retinal scans can be administered to a hospitalized patient as a part of screening for DVT condition which may pose a risk of developing into a potentially fatal pulmonary embolism, heart attack or cerebral stroke. Analysis of the retinal scans in the context of the patient's medical records may be used to determine a risk score for DVT and inform caregivers of the need for preventative or investigative measures as follow-up care.

In an example of the present disclosure, a method includes receiving an image of a retina of an eye of a patient, receiving patient data corresponding to the patient from an electronic medical record (EMR) of the patient, and determining, by a processor, a feature in the image. The method also includes determining, by the processor and by inputting the feature and at least a portion of the patient data as input to a machine learning (ML) model, a risk level associated with a first condition, determining, by the processor and based on the risk level being higher than a threshold, a recommendation for screening of the patient for the first condition, and providing, by the processor and to an output device, an output indicating the recommendation.

In another example of the present disclosure, a system includes memory, a processor and computer-executable instructions stored in the memory and executable by the processor. The instructions, when executed, cause the processor to perform operations comprising: receiving an image of a retina of an eye of a patient, receiving, from an electronic medical record (EMR) of the patient, patient data corresponding to the patient, determining a feature in the image, determining, by inputting the feature and at least a portion of the patient data as input to a machine learning (ML) model, a risk level associated with a first condition, determining, based on the risk level being higher than a threshold, a recommendation for screening of the patient for the first condition, and providing, to the EMR of the patient, an output indicating the recommendation.

In still another example of the present disclosure, a non-transitory computer-readable storage medium storing processor-executable instructions that, when executed, cause one or more processors to: receive, from an optical imaging device, an image of a retina of an eye of a patient, access, from an electronic medical record (EMR) storage, EMR data of the patient, determine a feature in the image, determine, by inputting the feature and at least a portion of the EMR data as input to a machine learning (ML) model, a risk level associated with a first condition, and determine, based on the risk level being higher than a threshold, a recommendation for screening of the patient based on the first condition.

### BRIEF DESCRIPTION OF THE DRAWINGS

Features of the present disclosure, its nature, and various advantages, may be more apparent upon consideration of the following detailed description, taken in conjunction with the accompanying drawings.

FIG. 1 illustrates an example environment for screening for a risk of a DVT condition based on a combination of retinal scan(s) and health records of a patient.

FIG. 2 illustrates a first block diagram of an example process for analyzing features of the retinal scans, and generating a recommendation based on risk of DVT, as described herein.

FIG. 3 illustrates a second block diagram of an example process for determining a risk assessment of DVT condition using a transformer-based architecture, as described herein.

FIG. 4 provides a first flow diagram illustrating an example method of the present disclosure for generating a recommendation for the patient.

FIG. 5 provides a second flow diagram illustrating an example method of the present disclosure for training an AI system for generating a recommendation for the patient.

FIG. 6 illustrates a schematic view of an example device configured to enable and/or perform the some or all of the functionality discussed herein.

In the figures, the left-most digit(s) of a reference number identifies the figure in which the reference number first appears. The use of the same reference numbers in different figures indicates similar or identical items or features. The drawings are not to scale.

### DETAILED DESCRIPTION

The present disclosure is directed, in part, to a risk assessment system for DVT, which may be programmed or otherwise configured to generate a recommendation for further screening, and corresponding methods. Such an example risk assessment system may be configured to accept, as inputs, one or more retinal images of a patient, and the patient's electronic health records, and generate, as output, the recommendation for further screening. The risk assessment system of the present disclosure may be included in regular care and monitoring of hospitalized patients. For example, retinal images of the patients may be captured using a vision screening device at regular intervals (e.g., daily) and provided as inputs to the risk assessment system. The risk assessment system may determine, based on analysis of the retinal images and information in the health records, a risk level associated with DVT, and provide, based on the risk level, a recommendation for further screening to verify and/or treat the DVT condition. The risk assessment system may make the recommendation available to a clinician caring for the patient.

In some examples, the present disclosure is directed to methods for screening for blood clot-related conditions that develop in hospitalized patients that may help in prevention of death due to pulmonary embolism (due to a blood clot reaching the lung), heart attack (due to a blood clot reaching the heart) or a cerebral stroke (due to a blood clot reaching the brain). Based on the risk level associated with the DVT condition, the methods of the present disclosure may recommend further screening tests or treatment, which may include D-dimer tests, imaging of blood vessels, administering blood thinning or thrombolytic medication, and the like. In some examples, the risk assessment system may generate, based on the risk level exceeding a threshold, a warning of a danger to the patient requiring urgent attention to prevent fatality.

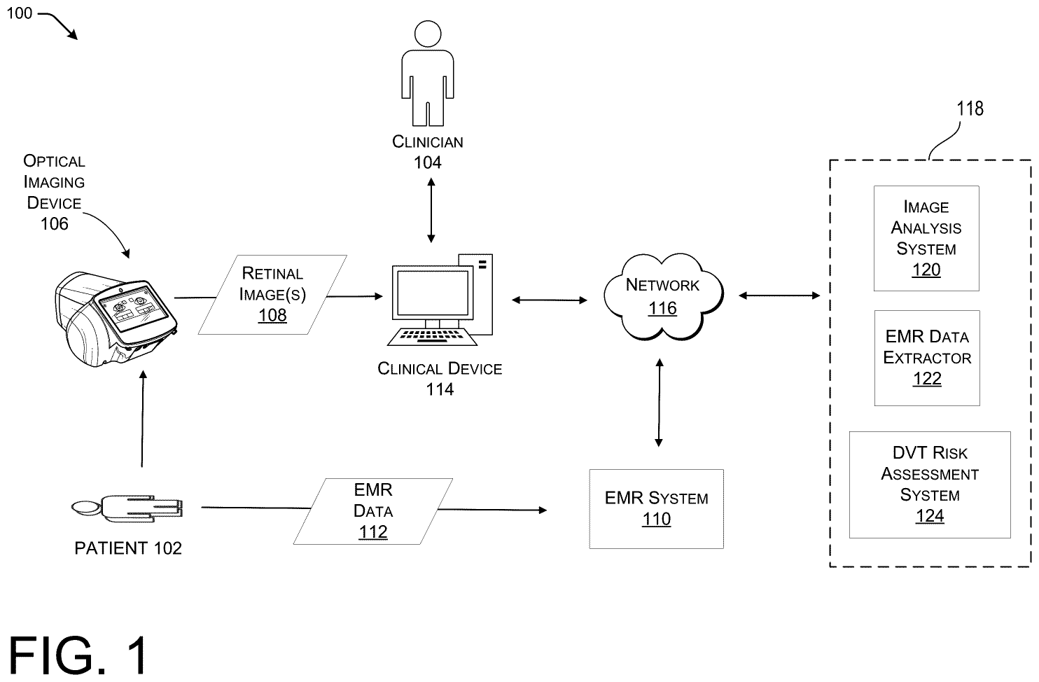

In some examples, the risk assessment system may determine a risk for the DVT or PE conditions by using trained machine-learned (ML) model(s) or other AI techniques which have been trained on anonymized data from large numbers of patient health records, including the patient's medical history and diagnoses, outcomes, medical test results, and retinal images captured during their hospital stay. In examples, the risk assessment system may leverage artificial intelligence (AI)-generated correlations between features of retinal images and information in the patient's health record, including the patient's medical history, medical test results and trends in such records. In some examples, the system may include detectors for specific features in retinal images (e.g., cotton wool spots, Roth spots, retinal vein occlusion (RVO), retinal vessel emboli, etc.) using image processing techniques applied to the retinal images (or scans). Various implementations of the present disclosure will be described in detail with reference to FIGS. 1-6. It is to be appreciated that while these figures describe methods and systems of the present disclosure, any examples set forth in this specification are not intended to be limiting and merely set forth some of the many possible implementations.

FIG. 1 illustrates an example environment 100 for screening a patient 102 to assess risk of blood-clot related conditions in the patient 102. In examples, the patient 102 may be any individual who is hospitalized and/or immobile in a hospital bed e.g., due to a recent surgery or disease-related condition, being monitored in a clinical environment and under the care of a clinician 104 e.g., a doctor, a nurse, etc. The screening may be based at least in part on imaging an eye of the patient 102 using an optical imaging device 106.

In examples, the optical imaging device 106 may be configured to obtain one or more images 108 of a retina and/or a fundus (which includes a back surface of an eye comprising the retina, macula, optic disc, fovea, and blood vessels) of at least one eye of the patient 102. In various implementations, the optical imaging device 106 may include an optical coherence tomography (OCT) camera configured to obtain OCT or OCT angiography (OCTA) images of the eye(s) of the patient 102. In some cases, the optical imaging device 106 may comprise a slit lamp imaging device configured to obtain slit lamp images (or projection images) of the eye(s) of the patient 102. In some examples, the optical imaging device 106 may include at least one fluorescence camera configured to obtain one or more fluorescence angiograms of the eye(s) of the patient 102. In some examples, the optical imaging device 106 may be configured to generate one or more color fundus (e.g., retinal) photography (CFP) images of the eye(s) of the patient 102, as described in U.S. patent application Ser. No. 18/099,062, filed Jan. 19, 2023, titled “Vision Screening Device Including Color Imaging,” which is hereby incorporated by reference in its entirety and for all purposes. The optical imaging device 106 may also capture one or more fluorescein angiography (FA) images of the eye(s) of the patient 102, one or more indocyanine green (ICG) angiography images of the eye(s) of the patient 102, one or more fundus autofluorescence (FAF) images of the patient, or any combination thereof.

The environment 100 may also include an electronic medical record (EMR) system 110 configured to store EMR data 112 associated with the patient 102. As used herein, the terms “electronic health record,” “electronic medical record,” “EMR,” and their equivalents, may broadly refer to stored data, in any modality of storage (e.g., temporary, transitory, permanent, etc.), indicative of a medical history and/or medical condition(s) of an individual, wherein the stored data is accessible (e.g., can be modified and/or retrieved) by one or more computing devices. EMR data of an individual may include their medical history indicating previous or current medical diagnoses, diagnostic tests, or treatments of the individual. In addition, the EMR data may indicate demographics of the individual (e.g., age, sex, race, etc.), parameters (e.g., vital signs, blood pressure, body mass index (BMI), etc.) of the individual, lifestyle information (e.g., smoking or drug use status, physical activity level, diet, alcohol use, etc.), notes from one or more medical appointments attended by the individual, medications prescribed or administered to the individual, therapies (e.g., surgeries, outpatient procedures, etc.) administered to the individual, results of diagnostic tests performed on the individual, identifying information (e.g., a name, birthdate, etc.) of the individual, or a combination thereof. For the example patient 102, who is hospitalized, the EMR data 112 may also include information related to the hospitalization e.g., details of surgical procedure or illness, health status updates during the current hospital stay, medical tests performed and test results, and the like. In some examples, the EMR system 110 may be implemented on one or more servers, such as servers located at a data center.

In some examples, the EMR system 110 may be connected to a clinical device 114 via a network 116. The clinical device 114 can include a computing device, such as a device including at least one processor configured to perform operations. In some cases, the operations are stored in memory in an executable format. Examples of computing devices include a personal computer, a tablet computer, a smart television (TV), a mobile device, a mobile phone, or an Internet of Things (IoT) device. In some examples, the clinical device 114 may be monitored by the clinician 104 to check a health status of the patient 102 and/or the clinical device 114 may send a notification to the clinician 104 (e.g., to a smartphone, a tablet computer, and/or other mobile device) with a recommendation related to the patient 102. The clinical device 114 may provide a user interface to the clinician 104 e.g., to access or enter data related to the patient 102, receive recommendation(s) from a risk assessment system, and/or view the retinal image(s) 108 captured by the optical imaging device 106. In examples, the clinical device 114 or the optical imaging device 106 may store the retinal image(s) 108 of the eye(s) of the patient 102 in the EMR system 110 in association with the EMR data 112 of the patient 102.

In examples, the network 116 may represent one or more communication networks. Examples of communication networks include at least one wired interface (e.g., an ethernet interface, an optical cable interface, etc.) and/or at least one wireless interface (e.g., a BLUETOOTH interface, a WI-FI interface, a near-field communication (NFC) interface, a Long-Term Evolution (LTE) interface, a New Radio (NR) interface, etc.). In some examples, data or other signals may be transmitted between elements of FIG. 1 over a wide area network (WAN), such as the Internet. In some cases, the data may include one or more data packets (e.g., Internet Protocol (IP) data packets), datagrams, or a combination thereof.

In various examples, the clinical device 114 may be connected, via the network 116, to a remote computing device 118, such as a server implemented on a cloud platform. In examples, the clinical device 114 may upload, to the remote computing device 118, the retinal image(s) 108 captured by the optical imaging device 106. The remote computing device 118 may implement an image analysis system 120 that receives, as an input, the retinal image(s) 108 captured by the optical imaging device 106 and analyze the image(s) 108 to determine various features. In other examples, the optical imaging device 106 may be in direct communication with the remote computing device 118 to upload the retinal image(s) 108, and/or the image analysis system 120 may be implemented, completely or in part, on the clinical device 114.

In some examples, the image analysis system 120 may implement one or more image processing components to identify locations of significant landmark(s) in the retinal image(s) 108 of the eye(s). For example, the locations may be indicated by identifying pixels in the input image that correspond to an area of the respective landmark. As used herein, the term “landmark,” and its equivalents, may refer to an anatomical structure that is observed in healthy or diseased eyes. Examples of landmarks include one or more of a macula, an optic disc (OD), a retina, a cornea, an iris, a lens, one or more retinal layers, one or more blood vessels, or a fovea. In some examples, the image analysis system 120 may implement image processing techniques, such as edge detection and connected component analysis, to determine portions of the retinal image(s) 108 corresponding to blood vessels in the eye, and determine characteristics (e.g., color, sharpness, continuity, etc.) associated with the blood vessels. The one or more image processing components may comprise first machine learning (ML) models configured to output a location of one or more of a set of landmarks in an input image of the back of the eye(s).

In some examples, the image analysis system 120 may implement feature detectors to identify different types of ophthalmic features in the image(s) 108. As used herein, the term “feature,” and its equivalents, may refer to a structure or visible sign within an image of an eye that may be correlated with one or more diseases and/or medical conditions. Examples of ophthalmic features may include one or more of a cotton wool spots (CWS), Roth spots, retinal vessel emboli, retinal vein occlusion (RVO), microaneurysm, a hemorrhage, drusen, exudate, edema, a cup/disc ratio (CDR), focal arteriolar narrowing, arterio-venous nicking, a red spot, retinal whitening, a Hollenhorst plaque, a microinfarct, coagulated fibrin, new vessels elsewhere (NVE), a vitreous hemorrhage (VH), a pre-retinal hemorrhage (PRH), new vessels on a disc (NVD), venous beading, an intraretinal microvascular abnormality (IRMA), diameter and topology of blood vessels, retinal vascular caliber, average diameter of retinal arterioles and venules summarized as arteriovenous ratio (AVR), etc.

In some examples, one or more of the feature detectors may comprise second machine learning (ML) models configured to output a binary indication of whether the respective feature is present or not along with a confidence score, and/or a location of the feature in an input image. Additionally, the image analysis system 120 may output various characteristics of the detected ophthalmic features, such as the location of the feature in the image (e.g., a quadrant of the eye the feature is located, a distance and/or direction from one or more of the landmarks, a proximity to the one or more landmarks, and the like), and a size of the feature in the image(s) (e.g., relative to size(s) of the landmark(s), as a number of pixels, as a fraction of area of the feature of the total retinal area, etc.). In examples, the image analysis system 120 may also implement image processing techniques to detect the features and/or determine one or more measurements associated with the detected ophthalmic features e.g., diameter of a blood vessel, length of blood vessel, occlusions on blood vessel, number of spots, density of spots, textural elements of the feature, etc.

In some examples, the image analysis system 120 may also determine ophthalmic features indicating color(s) associated with the landmarks in the eye. For example, the image analysis system 120 may determine ophthalmic features indicating an average color value (e.g., in a color space such as RGB, HSI, CIE L*a*b*, CIE L*u*v*, etc.) of the blood vessels, the fovea, optic disc, or the retina. The image analysis system 120 may also determine ophthalmic features indicating relative intensities between various landmarks or areas of the fundus.

Additionally, in some examples of the present disclosure, the image analysis system 120 may generate a standardized retinal image from the retinal image(s) 108 captured by the optical imaging device 106. In examples, the image analysis system 120 may generate the standardized retinal image by scaling (e.g., to a standard size and aspect ratio) and normalizing (e.g., histogram stretching to cover full range of brightness and contrast levels) the image(s) captured by the optical imaging device 106, such that the landmarks of the retina (e.g., optical disc, fovea, important blood vessels, etc.) are located at pre-determined positions relative to an image boundary of the standardized retinal image. For example, after such standardization, the landmarks may be in alignment in multiple different standardized retinal images (e.g., occurring at a same location relative to the respective image boundaries). In examples, the image analysis system 120 may associate a timestamp indicating a date/time of capture of the image(s) 108 with each of the ophthalmic features and the standardized retinal image determined from the image(s) 108.

In examples, the first ML models and the second ML models of the image analysis system 120 may be pre-trained based on training images e.g., from a training dataset. For example, the first ML models may be trained on a first training dataset including images of the back of the eye(s) (e.g., retinal images) labeled with the set of landmarks, and the second ML models may be trained on a second training dataset, the second training dataset including example images depicting the ophthalmic features, as well as ground truth information associated with each image identifying the ophthalmic feature depicted therein. In examples, one or more expert annotators may review the images in the training datasets and indicate, as ground truth information, the landmarks and/or whether the respective images depict one or more of the ophthalmic features.

In examples, the remote computing device 118 may also implement an EMR data extractor component 122. The EMR data extractor component 122 may extract demographic data (e.g., age, sex, race, etc.), health parameters (e.g., vital signs, blood pressure, body mass index (BMI), etc.), lifestyle information (e.g., smoking or drug use status, physical activity level, diet, alcohol use, etc.), geographic region of residence, previous or current medical diagnoses, diagnostic tests and results, medical treatments, prescriptions, etc. The EMR data extractor component 122 may also ignore some data of the EMR data 112 (e.g., a name, address, emergency contact, etc.) based on irrelevance to disease status. In some examples, the EMR data extractor component 122 may process the EMR data 112 to extract data of interest for assessing risk of DVT, which may include data related to risk factors for venous stasis, endothelial damage, and/or hypercoagulability risk, which are correlated with the risk of DVT. In particular, the data of interest may include information related to the current hospital stay of the patient 102, such as reason for hospital stay, duration of stay, and/or test results, and risk factors associated with DVT. Some examples of EMR data associated with DVT risk factors are summarized below in Table 1.

TABLE 1

| EMR data indicating | EMR data indicating | EMR data indicating |
|---|---|---|
| Venous Stasis risk | Endothelial Damage risk | Hypercoagulability risk |
| Sedentary lifestyle | Non-obstructive | Recent major trauma or |
| | cardiovascular disease | surgery |
| Hypertension | Hypertension | Immobilization or prolonged |
| | | hospital stay |
| Kidney failure | Hyperglycemia | Disease diagnoses such as |
| | | cancer, autoimmune diseases, |
| | | sepsis or heart infection |
| Multiple pregnancies | Hyperlipidemia | Hormonal contraception use |
| Obesity | Physical inactivity | Obesity |
| Heart disease | High cholesterol levels | Genetic factors |
| Varicose veins | Age | Smoking |

In examples, the EMR data extractor component 122 may convert the data of interest from the EMR data 112 to a pre-defined standard representation. As an example, the EMR data extractor component 122 may create a vector (e.g., an EMR feature vector), which may be a one or two-dimensional array with data fields indicating the data of interest from the EMR data 112, and associate a timestamp with each data field indicating a date/time when the data of interest was determined or added to the EMR data 112. In some examples, the EMR data extractor component 122 may include, in the EMR feature vector, a first set of data from the EMR data 112 in their respective native form (e.g., text specifying disease condition(s), numerical values specifying age, BMI number, blood glucose level, and the like), represent a second set of data as levels (e.g., 1-3, 1-5, 1-10, etc.) corresponding to ranges of values (e.g., 1: low, 2: medium, 3: high), and represent a third set of data (e.g., textual data) as category numbers (e.g., 0: non-smoker, 1: smoker in a “smoking status” data field, 0: sedentary, 1: low activity, 2: medium activity, 3: active in an “activity level” data field, and so on). In some examples, the EMR data extractor component 122 may also divide numerical values of the EMR data 112 into range levels and enter the respective range level in the EMR feature vector. For example, an age of a patient may be divided into 1: under 2, 2: 2-5 years, 3: 5-11 years, 4: 12-18 years, 5: 18-54 years, and 6: 55+ years, where the EMR feature vector includes a range level indicator (1-6) corresponding to the age of the patient 102.

In examples, the remote computing device 118 may also implement a DVT risk assessment system 124 which may use various AI (e.g., artificial intelligence-based) techniques to generate recommendations for screening the patient 102 for DVT based on the retinal image(s) 108 and the EMR data 112. For example, the image analysis system 120 may provide the detected ophthalmic features and/or the standardized retinal image, as inputs, to the DVT risk assessment system 124, and the EMR data extractor 122 may provide the representation of the EMR data 112, as inputs, to the DVT risk assessment system 124, as inputs. For example, the ophthalmic feature retina vein occlusion (RVO) is a block in one of the small arteries or veins that carry blood to and from the retina in a patient's eye. An occlusion in one of these blood pathways may be an indicator of clots elsewhere in the body. Therefore, a finding of RVO in the retinal image(s) 108, in conjunction with EMR data 112 features such hypertension and recent surgery (which are risk factors, as shown in Table 1) may indicate an elevated risk level for DVT.

In examples, the DVT risk assessment system 124 may be configured to determine a risk level indicating risk of the patient 102 for developing a DVT condition, which may lead to a potentially fatal pulmonary embolism, heart attack or cerebral stroke. In various examples, the DVT risk assessment system 124 may use, as inputs, the retinal image(s) 108 captured by the optical imaging device 106, the ophthalmic features and/or standardized images determined by the image analysis system 120, the EMR data 112 associated with the patient 102, or a combination thereof, to determine the risk level for DVT exhibited by the patient 102. In examples, the DVT risk assessment system 124 may receive the EMR data 112 associated with the patient 102, from the clinical device 114 and/or directly from the EMR system 110.

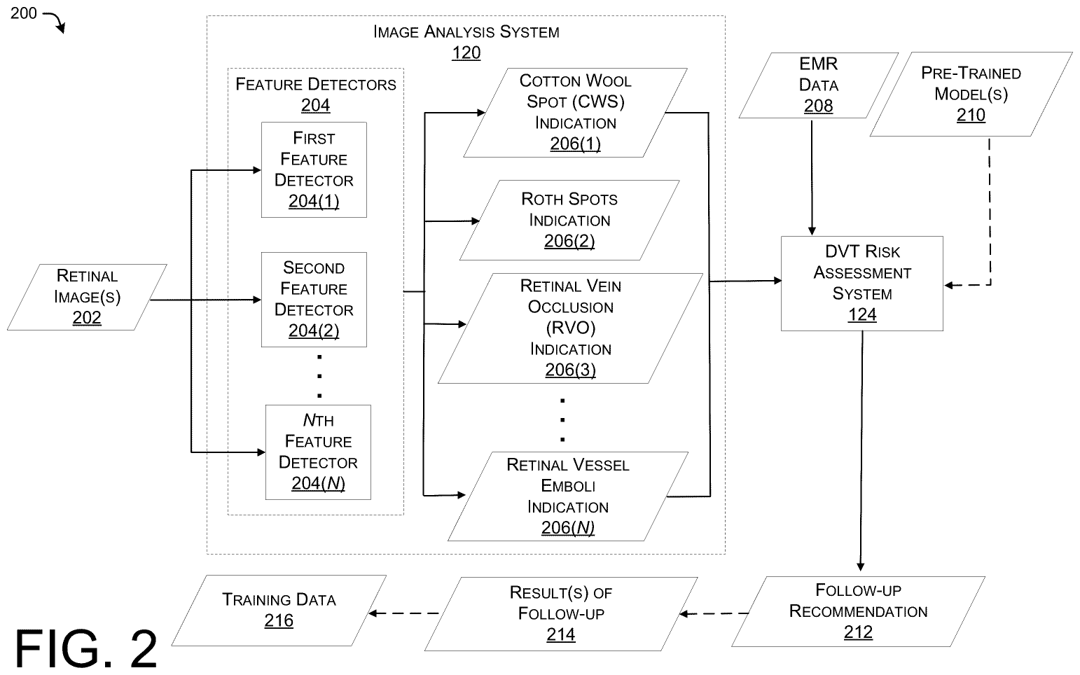

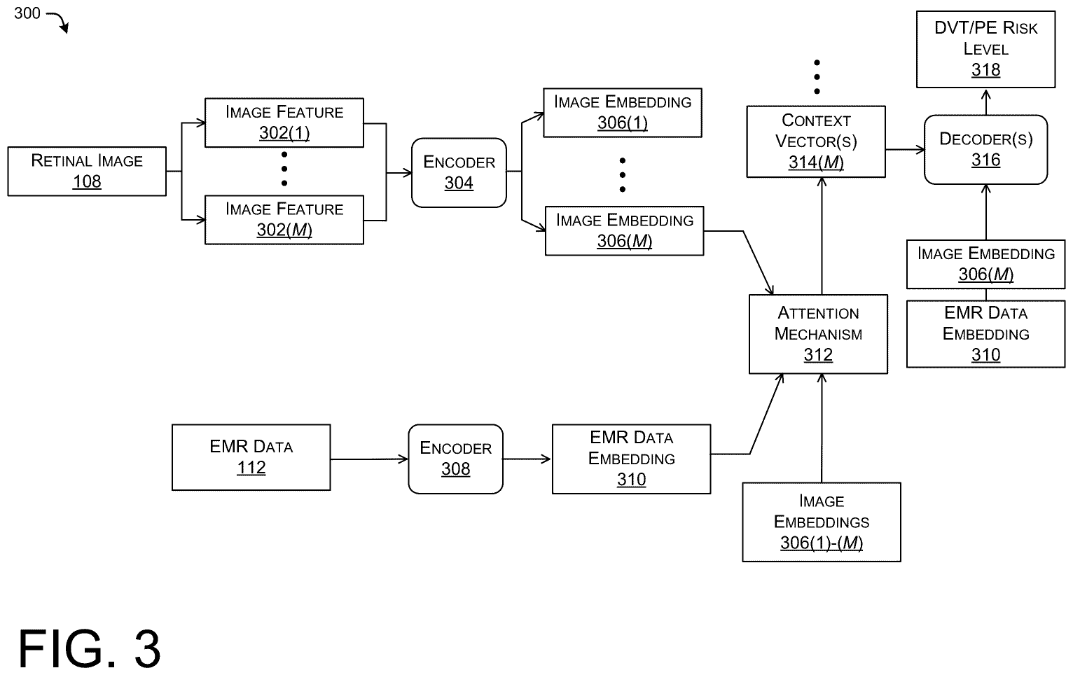

In examples of the present disclosure, the DVT risk assessment system 124 may comprise machine-learned (ML) models, image processing systems in conjunction with expert systems, statistical models, and the like, as described in further detail with reference to FIGS. 2 and 3. Machine learning models of the DVT risk assessment system 124 may be trained on large sets of anonymized training data comprising patient health records, including patient outcomes, diagnoses and medical test results during a period of hospital stay, EMR data of the patients, and corresponding retinal images taken at regular intervals (e.g., daily). Such training data may be accumulated from patient data in the EMR system 110 and/or data from publicly available health studies e.g., heart disease risk studies, sleep apnea studies, diabetes risk studies, etc. Examples of using machine learning techniques for disease and health risk determination from retinal images and EMR data of the patient are described in U.S. Patent Application Ser. No. 63/601,463, filed Nov. 21, 2023, titled “Automated Disease Detection using Retinal Images,” which is hereby incorporated by reference in its entirety and for all purposes.

In examples, the DVT risk assessment system 124 may generate a recommendation based on the risk level for the patient 102 developing a DVT condition in the near future. For example, the recommendation may indicate that the patient requires further screening, or indicate that the screening was normal (e.g., no elevated risk of DVT). In some examples, the recommendation may also include an associated urgency level e.g., follow-up screening on same day, screening tests within 3 days, immediate attention needed or life-threatening condition suspected, etc., based on a determined risk level. In some examples, the DVT risk assessment system 124 may transmit the recommendation to the clinical device 114, where the recommendation may be output, via a user interface, to the clinician 104. Accordingly, the clinician 104 may take various actions to schedule follow-up tests, prescribe preventative medications, and/or add the recommendation to the EMR data associated with the patient 102. In some examples, the DVT risk assessment system 124 may transmit the recommendation to the EMR system 110. In such examples, the recommendation may be accessed from the EMR system 110 by other users (e.g., physicians, nurses, etc.) caring for the patient 102.

As used herein, the terms “machine learning,” “ML,” and their equivalents, as used with reference to the image analysis system 120 and the DVT risk assessment system 124, may refer to a computing model that can be optimized to accurately recreate certain outputs based on certain inputs. In some examples, the ML models include deep learning models, such as convolutional neural networks (CNN) or recurrent neural networks (RNN), transformer architectures, any combination thereof, or other types of NNs. The output of the ML models can be in any form based on the purpose of the learning network. For example, the output can be a name of a detected feature, a location of the detected feature, an indication of the presence of the detected feature, a probability or confidence score of the presence of a detected feature, a risk level (e.g., a real number between 0 and 1, a percentage, a score between 0 and 10, etc.), and the like.

It should be understood that, while FIG. 1 depicts a single optical imaging device 106, in additional examples, the environment 100 may include any number of local or remote optical imaging devices configured to operate independently and/or in combination to capture various types of retinal images. Additionally, although FIG. 1 illustrates the optical imaging device 106, the EMR system 110, the clinical device 114, and the remote computing device 118 as separate entities, in some implementations, one or more of these entities may be correspond to the same computing device.

As discussed herein, FIG. 1 depicts an exemplary environment 100 that includes components for capturing retinal images of a patient, and based on the retinal images and additional information from health records of the patient, providing, by a trained AI-based DVT risk assessment system, recommendation(s) for further screening the patient for blood-clot related risks. The environment 100 enables monitoring of a hospitalized patient for risk of developing DVT/PE based on a non-invasive retinal scan, and data available in the patient's medical records. The trained AI-based DVT risk assessment system may be continuously improved by re-training based on new training data instances, as described below.

FIG. 2 illustrates an example system 200 for generating a recommendation for further screening of a patient for DVT, where the recommendation is based on features of the retinal image(s) 108 and the EMR data 112. As illustrated, the environment 200 includes the image analysis system 120 and the DVT risk assessment system 124 described above with reference to FIG. 1.

As described above with reference to FIG. 1, the image analysis system 120 may receive and/or identify (e.g., from a computer memory) one or more retinal image(s) 202, which may be identical to the retinal image(s) 108 is some examples. In other examples, the retinal image(s) 202 may be a subset of the retinal image(s) 108. For example, some of the retinal image(s) 108 may be filtered out based on inadequate image quality, near-duplicates may be removed, a sampling of the retinal image(s) may be included in the subset (e.g., the retinal image(s) 108 may be captured daily, whereas the subset may include retinal image(s) from alternate days), and the like. In some examples, the retinal image(s) 108 may be enhanced via image processing techniques to obtain the retinal image(s) 202 e.g., via histogram equalization, contrast and/or brightness adjustments, image sharpening, edge enhancement, high-pass or low-pass digital filtering, etc. The image analysis system 120 may determine ophthalmic features, including structural and color features, of the retinal image(s) 202 as well as a standardized retinal image, as described with reference to FIG. 1. Some examples of ophthalmic features are described in U.S. patent application Ser. No. 17/709,950, filed Mar. 31, 2022, titled “Automated disease identification based on ophthalmic images,” which is hereby incorporated by reference in its entirety and for all purposes.

In some examples, the image analysis system 120 may include feature detectors 204 comprising a set of feature detectors e.g., first feature detector 204(1), second feature detector 204(2), . . . , N^{th} feature detector 204(N), configured to detect first to N^{th} ophthalmic features respectively in the retinal image(s) 202. Individual feature detectors 204 (1, . . . , N) may each generate, as output, an indication of the respective ophthalmic features when such features are present in the retinal image(s) 202 provided as input to the feature detectors 204. By way of example and not limitation, the first feature detector 204(1) may generate, as output, a cotton wool spot (CWS) indication 206(1), the second feature detector 204(2) may output a Roth spots indication 206(2), the third feature detector 204(3) may output a retinal vein occlusion (RVO) indication 206(3), and the N^{th} feature detector 204(N) may output a retinal vessel emboli indication 206(N). The feature detectors 204 may include detectors for various other features that may be observed in retinal images.

In examples, the ophthalmic features detected by the feature detectors 204 may correspond to features correlated with DVT e.g., ophthalmic features which may indicate presence of blood clots in the patient. As examples, CWS detected in a retinal image could indicate that the patient has hypertension or is in a hypercoagulable state, where the hypertension can be caused by blockage due to a blood clot. CWS may also be caused by an ischemic condition where blood flow is restricted or reduced, which may indicate a likelihood of blood clots. Roth spots may be indicative of infective endocarditis (e.g., due to a bacterial infection) which causes growths (or vegetations) on heart valves, producing toxins and enzymes that may cause holes in the valves, and can spread outside the heart and blood vessels. One of the complications of the vegetation is that embolisms can form from such growths and impede blood flow. Retinal vascular occlusive disease and anterior ischemic optic neuropathy may be seen in patients with atrial fibrillation who are typically at higher risk for PE and DVT. Another example of an ophthalmic feature correlated with blood clot formation is a presence of emboli in the retinal blood vessels.

In some examples, one or more of the feature detectors 204 may comprise a ML model trained to detect the respective ophthalmic feature in the retinal image(s) 108 provided as input to the ML model. In examples, the ML model may output characteristics associated with the respective ophthalmic feature, a location of the feature in the input retinal image, and/or a confidence level associated with the detection of the feature. As examples, the ML models corresponding to the individual feature detectors 204 may comprise CNNs, RNNs, fully convolutional networks (FCNs), transformer-based models, etc. For example, a dense output layer may be added to convolutional layers of a CNN or RNN, which outputs a binary determination of whether the respective ophthalmic features is present in the input retinal image or not, along with a confidence score of the determination.

In another example, one or more of the feature detectors 204 may comprise an FCN as the ML model. The FCN may be trained to generate, as output, an image of a same size as the input retinal image which indicates pixel locations in the output image that correspond to the respective ophthalmic feature. For example, in an FCN-based first feature detector 204(1), pixels in the output image corresponding to locations of regions of cotton wool spots in the input retinal image 202 may be of a high intensity value relative to pixels that do not correspond to regions of cotton wool spots.

In yet another example, one or more of the feature detectors 204 may comprise a transformer as the ML model. For example, in a transformer-based architecture, the retinal image(s) 108 may be represented as embeddings of portions (or patches) of the image(s) which include position encoding indicating positions of the portions (or patches) in the retinal image(s) 202 (e.g., with respect to the standardized retinal image), as described in further detail with reference to FIG. 3.

In examples, the ML models of the feature detectors 204 may be trained, during a training phase, using a training dataset of labeled retinal images. In examples, the training dataset may comprise retinal images illustrating examples of the ophthalmic features detected by the feature detectors 204 e.g., cotton wool spots, Roth spots, RVO, emboli, etc. Additionally, the training dataset may comprise retinal images illustrating examples of healthy eyes indicating an absence of the above ophthalmic features. For example, each data instance in the training dataset may comprise a retinal image, and a corresponding ground truth indicating a presence or absence of the ophthalmic features and/or an indication of locations (e.g., by highlighting pixels or providing bounding boxes) of the ophthalmic features. The retinal images of the training dataset may also include indications of locations corresponding to landmarks in the eyes (e.g., fovea, optic disc, macula, etc.). In some examples, the retinal images of the training dataset may also include ground truth indicating blood vessels in the retinal images.

In some examples, a training component (not shown) may train a set of ML models corresponding to one or more of the feature detectors 204, each ML model trained to output an indication of a specific ophthalmic feature related to risk of DVT (e.g., corresponding to the ophthalmic feature indications 206(1, . . . , N)), where the indication may comprise a confidence level associated with a presence of the feature, location(s) of the feature in the input retinal image 108, and/or characteristics of the feature, such as measurements, color values, intensity values, density, etc. The training component may train each such individual feature detector using a subset of the training dataset. For example, the first feature detector for detecting cotton wool spots (CWS) may be trained using a first subset of instances of the training dataset that indicates a presence of CWS and, as negative examples, a second subset of instances of the training dataset that indicates an absence of CWS.

In some examples, one or more feature detectors 204 may use pre-trained ML model(s) trained to identify general landmarks in retinal images (e.g., fovea, optic disc, macula, etc.). For example, such a pre-trained model may be trained using a large number of retinal images to identify landmarks in the retinal images. As can be understood, the retinal images for training the pre-trained model to identify landmarks may not require the retinal images to illustrate disease conditions and/or be labeled with ground truth indicating a disease condition, and therefore, be more readily available. The retinal images used for training the pre-trained model may be labeled with landmarks and/or disease features, or may be unlabeled. For example, the pre-trained ML model(s) may comprise unsupervised models, such as an autoencoder or a generative adversarial network (GAN), which may be obtained by unsupervised pretraining using unlabeled retinal images as training data. In such examples, the feature detectors 204 may utilize the pre-trained ML model(s) (e.g., lower layers of the trained autoencoder or the lower layers of the trained GAN) along with additional output layer(s) which may be trained using supervised learning with a smaller set of labeled training data. In some examples, the one or more feature detectors 204 may comprise ML model(s) obtained by refining (e.g., using transfer learning techniques) the pre-trained ML model(s) to detect specific features (e.g., CWS, Roth Spots, RVO, etc.) by using a smaller number of retinal images labeled with the respective features.

Alternatively, or in addition, one or more of the feature detectors 204 may detect ophthalmic features in the retinal image(s) 202 by applying image processing techniques on the retinal image(s) 202. By way of example and not limitation, CWS, Roth spots, and emboli in retinal blood vessels may be detected based on a combination of color information and shape of color regions. For example, pixels of the retinal image(s) 202 may be assigned to bins based on their color values e.g., a color space such as RGB, HSI, CIE L*a*b*, CIE L*u*v*, etc. may be binned into a discrete number of color values. Connected component analysis may be applied to pixels within a same bin or neighboring bins to determine regions of a similar color in the retinal image(s) 202. In examples, candidate regions corresponding to CWS spots may be characterized by whitish color and indistinct boundaries, candidate regions corresponding to Roth spots may be characterized by a red color value and distinct boundaries, and candidate regions corresponding to emboli may be characterized by whitish or yellowish segments surrounded by regions of dark red (which correspond to blood vessels). In some examples, edge detection techniques may be applied to the retinal image(s) 202 to further indicate the boundaries of regions.

In some examples, the feature detectors 204 may detect the ophthalmic features in the retinal image(s) 202 by comparing an appearance of the candidate regions corresponding to an ophthalmic feature with known (or labeled) retinal image(s) illustrating the respective ophthalmic feature e.g., the labeled retinal image(s) may indicate regions corresponding to the respective ophthalmic features. For example, the comparison may include a similarity in color, shape, numbers, or extent of the candidate regions with the regions indicated in the labeled retinal image(s).

In some examples, locations of candidate regions with respect to landmarks in the retinal image(s) 202 (e.g., fovea, optic disc, macula, blood vessels, etc.) may be used to determine the ophthalmic features. For example, central retinal vein occlusion (CRVO) and branch retinal vein occlusion (BRVO) may appear as clusters of regions (corresponding to hemorrhage) along locations of blood vessels. As another example, ischemic optic neuropathy may appear as regions proximate to the optic disc.

The feature detectors 204 may use image processing techniques, ML models, or a combination of both. In examples where a sufficient number of training data instances may not be available for training an ML model to detect an ophthalmic features, image processing techniques based on an appearance of the ophthalmic feature in the retinal image(s) (e.g., color, shape of region, spatial relationship to landmarks, etc.) may be used to implement a feature detector for the ophthalmic feature. In other examples, the feature detectors 204 may use apply image processing techniques to enhance and/or identify candidate ophthalmic features before providing the retinal image(s) 202 including the enhanced and/or identified candidate ophthalmic features as input to a ML model.

In examples, the image analysis system 120 may output a confidence score or probability of each indication 206 (1, . . . , N) e.g., as outputted by a respective ML model and/or image processing component of the feature detectors 204 (1, . . . , N). The confidence score may represent a level of certainty that the respective ophthalmic feature is detected in the input retinal image(s) 202, an amount or severity associated with the respective ophthalmic feature, and/or a quality of the input retinal image(s) 202.

In examples, the system 200 may provide the indications 206(1, . . . , N) of the image analysis system 120 and EMR data 208 to the DVT risk assessment system 124. The EMR data 208 may be the EMR feature vector output by the EMR data extractor component 122, as described with respect to FIG. 1, and may be identical to the EMR data 112 corresponding to the patient 102. In some examples, the EMR data 208 may comprise a text summary or a compilation of some or all of the EMR data 112 using output from pre-trained large-language models (LLMs). For example, some or all of the EMR data 112 may be provided as input prompts to LLMs to determine the EMR data 208.

In examples, the DVT risk assessment system 124 may be configured to determine a risk level of the patient 102 for developing a DVT or PE condition based on the EMR data 208 and/or the ophthalmic feature indications 206 exhibited by the retinal image(s) 202 of the patient 102. In examples of the present disclosure, the DVT risk assessment system 124 may use ophthalmic features indicative of blood clots in the patient 102 in conjunction with risk factors for developing blood clots indicated in the EMR data (as illustrated in Table 1) to determine the risk level of the patient 102. The DVT risk assessment system 124 may comprise machine-learned (ML) models, image processing systems in conjunction with expert systems, statistical models, and the like.

In some examples of the present disclosure, the DVT risk assessment system 124 may be trained on a training dataset comprising retinal images and EMR data of different individual patients (e.g., patients hospitalized due to surgery or illness), each patient corresponding to a data instance. In examples, the image analysis system 120 may process the retinal images in the training dataset to generate corresponding ophthalmic feature indications 206. The image analysis system 120 may also associate timestamps with the ophthalmic features indicating the date and/or time of day when the underlying retinal images were captured. The training dataset may also include ground truth indicating whether the patient developed DVT or PE during a period of time from a time of capture of the retinal image(s) of the patient. For example, the ground truth may indicate that the patient did not develop DVT or PE, or may indicate that the patient developed DVT or PE within the period of time (e.g., less than one day, one day, two days, five days, etc.) from the capture of the retinal image(s).

In some examples, large training datasets that include ground truth indicating a diagnosis of DVT or PE may not be available. However, some common health conditions (e.g., high blood pressure, diabetes, etc.) share similar indications in the retinal images (e.g., CWS, Roth Spots, etc.) as the DVT/PE condition. In some examples, the DVT risk assessment system 124 may use pre-trained ML model(s) 210 trained on labeled or unlabeled data, in conjunction with transfer learning, to determine the risk of developing DVT/PE. As an example, the pre-trained ML model(s) 210 may be trained to detect the common health conditions that share similar indications as DVT/PE, where training datasets may be more readily available. In some examples, the pre-trained ML model(s) 210 may be refined (or fine-tuned) using a small number of labeled training examples of DVT/PE (e.g., using a few-shot learning paradigm). As another example, the DVT risk assessment system 124 may use the pre-trained ML model(s) 210 in conjunction with additional rules (e.g., an expert system) to determine the risk of developing DVT/PE.

In some examples, the pre-trained ML model(s) 210 may comprise unsupervised models (e.g., an autoencoder, a GAN, a restricted Boltzmann machine (RBM), etc.), trained on unlabeled data (e.g., retinal images and corresponding EMR data without an indication of whether DVT/PE was present). In such examples, the DVT risk assessment system 124 may comprise an ML model that includes at least a portion of the pre-trained ML model(s) 210 (e.g., lower layers of the trained autoencoder or the lower layers of a discriminator of the trained GAN) along with additional output layer(s) which may be trained using supervised learning with a smaller set of labeled training data.

In some examples, the DVT risk assessment system 124 may use ML model architectures similar to those described with reference to the feature detectors 204 of the image analysis system 120 e.g., CNNs, RNNs, transformer-based models, etc. The ML model(s) of the DVT risk assessment system 124 may be trained to output a binary determination of whether a DVT condition is present or not, along with a confidence score associated with the determination. In such examples, the DVT risk assessment system 124 may map a higher confidence score output by the trained ML model with a higher DVT risk. As another example, a transformer-based ML model may be trained to output a predicted risk value for DVT based on the training dataset, as described with reference to FIG. 3.

In some examples of the present disclosure, the DVT risk assessment system 124 may comprise statistical models (e.g., Bayesian decision trees, regression models, etc.) determined from the training dataset by estimating conditional probabilities of DVT given the ophthalmic feature(s) detected and the EMR data of the patient. For example, a Bayesian decision tree may be constructed using Markov Chain Monte Carlo (MCMC) sampling of the training dataset. The DVT risk assessment system 124 based on statistical models may output a probability score of DVT, where a higher probability score indicates a higher risk of DVT. Additionally, an advantageous aspect of such statistical models relative to ML models is that the statistical models may provide an analysis of how the probability score was determined e.g., a probability of DVT associated with the ophthalmic feature(s) and/or EMR data considered by the DVT risk assessment system 124. In some examples, the DVT risk assessment system 124 system may comprise an expert system based on the underlying statistical model, which may be further refined by medical professionals experienced in diagnosing blood clot related conditions and/or diagnosing systemic health issues based on retinal image(s). In some examples, the DVT risk assessment system 124 system may comprise an expert system including rules provided by medical professionals or a hospital's protocols to determine a risk of DVT based on the indications 206 and the EMR data 208 (e.g., when a sufficient number of data instances is not available to train statistical models or ML models).

In examples, the DVT risk assessment system 124 may generate a follow-up recommendation 212 based on the risk level of developing DVT determined to be exhibited the patient 102. For example, the recommendation 212 may indicate that the patient requires additional screening, or indicate that there is no elevated risk of DVT condition. In examples, the determined risk level may be compared with a threshold, and based on the risk level being higher than the threshold, the DVT risk assessment system 124 may generate the recommendation 212 for further screening or treatment. Such further screening may include blood tests such as the D-dimer test or imaging tests such as MRI or ultrasound. The recommendation 212 may also trigger treatments such as preventative medications (e.g., thrombolytic medications or blood thinners). In some examples, the recommendation may also include an associated urgency level (e.g., follow-up immediately, within one day, within 3 days, within a week, etc.) based on comparing the determined risk level with thresholds indicating severity of the risk.

In some examples, there may be different standards of practice for follow-up care when DVT/PE condition is suspected, which may be based on a hospital's care protocol, or practices recommended in a country, geographic region, or an insurance policy. In various implementations, the DVT risk assessment system 124 may determine the recommendation 212 according to standards of practice followed in a hospital or location where the patient 102 is being treated. For example, the hospital where the patient 102 is being treated may implement an escalation protocol where a specialist may be called to check the patient 102 of the recommendation 212 indicates a risk of DVT/PE condition. In such an example, the recommendation 212 may include a check-up by a specialist.

In some examples, the DVT risk assessment system 124 may include, in the recommendation 212, a textual summary indicating findings in the indications 206 and/or the EMR data 208 that support the recommendation 212. For example, the DVT risk assessment system 124 may indicate rules that were satisfied in an expert system or statistical model, text from EMR data 208, positive or negative findings in the indication(s) 206, and the like, to support the recommendation 212.

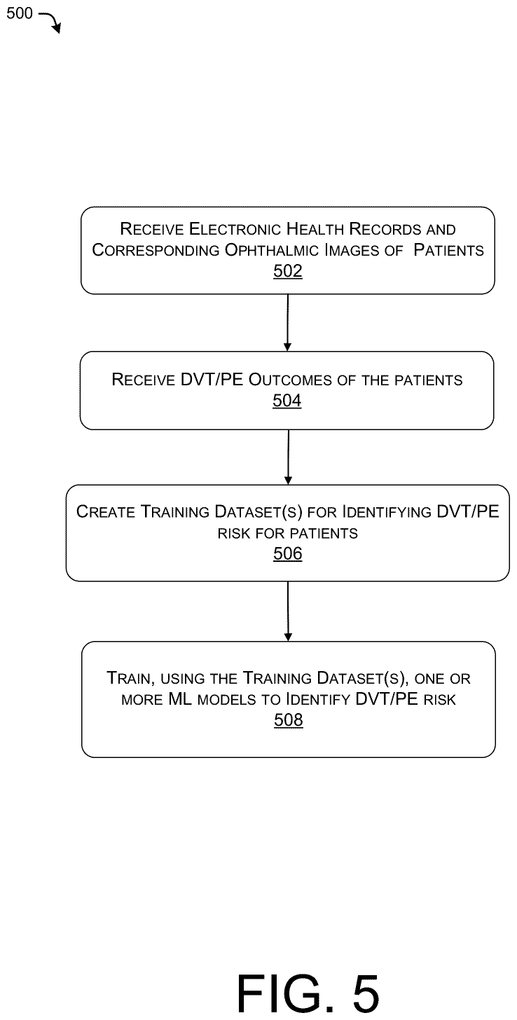

In some examples, the system 200 may include receiving result(s) of follow-up screening 214 based on the recommendation 212. In examples, the result(s) of follow-up 214 may be added as an instance of training data (e.g., training data 216) to a training dataset used to train the DVT risk assessment system 124 as discussed above, and described in further detail with reference to FIG. 5. In examples, the indications 206 and the EMR data 208 may be included in the training data 216 along with an indication of outcome. For example, if the result(s) of follow-up 214 indicate a presence of DVT or PE (e.g., a DVT or PE diagnosis is confirmed from an ultrasound scan or D-dimer test result), the training data 216 may indicate a true positive example, whereas if the result(s) of follow-up 214 do not indicate a presence of DVT, the training data 216 may indicate a false positive example (e.g., a negative example). In some examples, the result(s) of follow-up 214 may include inputs from medical professionals indicating the outcome or further comments related to the training data 216. In another scenario, if the follow-up recommendation 212 indicates no elevated risk of DVT, and the patient is later diagnosed with DVT or PE or patient death occurs due to a blood clot-related condition, the training data 216 may indicate a false negative example. In some examples, the training data 216 may be added to the training dataset only if the patient 102 (or a representative of the patient 102) has agreed to allow their data to be used in this manner (e.g., for addition to an anonymized training dataset). The DVT risk assessment system 124 may be re-trained using a training dataset augmented over time with false positive and false negative examples (e.g., by addition of instances of the training data 216), as discussed above, to refine the underlying machine-learned model, which may result in improved accuracy of the DVT risk assessment system 124 over time.

FIG. 3 illustrates an example process for determining a risk assessment for DVT condition using a transformer-based architecture. In examples, image features 302(1, . . . , M) may be generated from the retinal image 108. In some examples, the image features 302(1, . . . , M) may each comprise a patch or region of the retinal image 108, along with a position of the patch or region in the retinal image 108. For example, the retinal image 108 may be divided into a grid of M cells, with each image feature 302 representing a cell of the grid, where a position is indicated by (x, y) coordinates of the cell. In another example, the retinal image 108 may be segmented into regions based on consistency of color, texture, edge distribution, etc., where each image feature 302 may represent a region, where a position of the image feature is indicated by (x, y) coordinates of a centroid of the region. In examples, the (x, y) positions of the image features 302 may be encoded using a sine/cosine positional encoding to obtain position embeddings corresponding to each image feature 302. Alternatively, or additionally, in some examples, the retinal image(s) 202, which may be a subset of or enhanced version(s) of the retinal image(s) 108, may be used to generate the image features 302.

In examples, an encoder 304 may encode the image features 302(1, . . . , M) and associated positions into corresponding image embeddings 306(1, . . . , M). For example, the encoder 304 may project each image feature, such as the image feature 306(M), into an embedding space as an image embedding 306(M). The image embeddings 306 may be high-dimensional vectors or tensors that represent the image features 302 and associated positions in the embedding space. For example, each image embedding 306 may comprise the projection of the respective image feature 302 concatenated with the associated position embedding of the respective image feature.

Similarly, an encoder 308 may determine an EMR data embedding 310 based at least in part on the EMR data 112 for the patient 102 for whom a predicted DVT/PE risk is to be determined. The encoder 308 may project the EMR data 112 into an embedding space as the EMR data embedding 310. The embedding may be a high-dimensional vector or tensor that represents this data in the embedding space.

In examples of the transformer-based architecture, an attention mechanism 312 (such as self-attention or cross-attention) may be implemented that generates context vector(s) or attended context for each image embedding 306. For example, the attention mechanism 312 may output the context vector(s) 314(M) corresponding to the image embedding 306(M) based on the EMR data embedding 310 and all the image embeddings 306(1, . . . , M) generated from the retinal image 108. Though shown for an example image embedding 306(M), separate attention layers implementing the attention mechanism 312 may each generate a context vector(s) corresponding to each image embedding 306(1, . . . , M), to obtain context vectors 314 (e.g., context vector 314(1, . . . , M)) which may be provided to decoder(s) 316 to obtain an output associated with the given inputs (the retinal image 108 and the EMR data 112).

Standard attention mechanisms, including self-attention and cross-attention, may be applied at the encoder, decoder, or both, in a transformer-based architecture. In examples of a transformer-based architecture, computation of attention is expressed in terms of query, key, and value vectors, where the vectors are obtained by a vector-to-matrix multiplication of input embeddings with weight matrices which may be learned during a training phase. Typically, multiple query, key, and value vectors may be grouped together into matrices Q, K, and V corresponding to queries, keys, and values respectively. The embeddings selected for queries, keys, and values are based on the attention mechanism e.g., for self-attention the queries and keys may include embeddings generated for a same token, while cross-attention uses a query generated for a first token and a key generated for a second token.

As an example, for generating the context vector(s) 314(M), the attention mechanism 312 may use weighted EMR data embedding 310 as query matrix (Q) and weighted image embedding 306(M) as key matrix (K) to determine attention scores. The attention mechanism 312 may then scale and softmax the attention scores before computing a dot product with weighted image embeddings 306(1, . . . , M) (as value matrix (V)) to determine the context vector 314(M).

The context vector(s) 314 may indicate contextual information from the EMR data embedding 310 that may be relevant to interpreting the image embeddings 306 representing information in the retinal image 108 e.g., the EMR data embedding 310 may represent risk factors associated with DVT, that may be helpful in disambiguating image features caused by DVT from similar image features caused by other conditions such as hypertension or diabetes. The context vector(s) 314 may be provided to one or more decoder(s) 316, which may determine a predicted value of DVT/PE risk level 318 associated with the retinal image 108 and EMR data 112. In some examples, multiple context vectors 314 associated with different image features may be provided as input to the decoder 316 to determine the predicted DVT/PE risk level 318. In some examples, the decoder(s) 316 may use the context vector(s) 314 alone, the context vector 314 and EMR data embedding 310 and/or the image embedding 306(M) to determine the predicted value. Additionally or alternatively, in some examples, the decoder(s) 316 may include multiple multi-headed self-attention layers, and subsequent addition and normalization layers, in combination with a feedforward network (e.g., a multi-layer perceptron), for combining predicted values from different image embeddings 306.

The example transformer-based architecture illustrated in FIG. 3 is an example and not a limitation. It is understood that various other combinations of encoders, decoders and attention mechanisms are possible, and the present disclosure is intended to include variations to, and modifications of, the transformer-based architecture of FIG. 3.

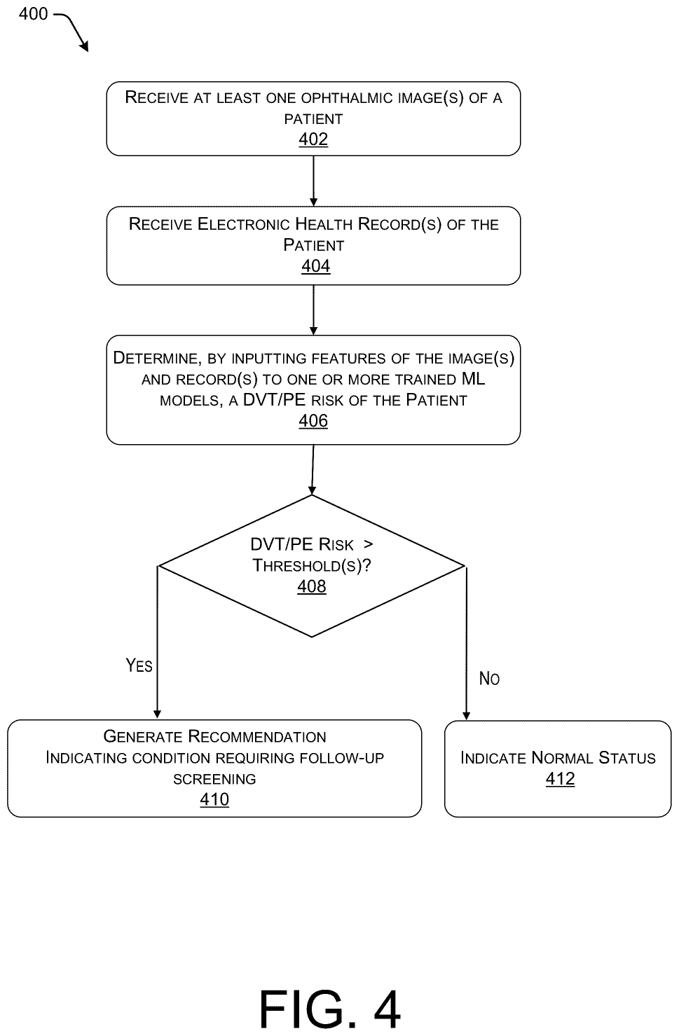

FIGS. 4 and 5 provide flow diagrams illustrating example methods for generating a recommendation for screening of disease(s), as described herein. The methods in FIGS. 4 and 5 are illustrated as collections of blocks in a logical flow graph, which represents sequences of operations that can be implemented in hardware, software, or a combination thereof. In the context of software, the blocks represent computer-executable instructions stored on one or more computer-readable storage media that, when executed by processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order and/or in parallel to implement the methods illustrated in FIGS. 4 and 5. In some embodiments, one or more blocks of the methods illustrated in FIGS. 4 and 5 can be omitted entirely.

The operations described below with respect to the methods illustrated in FIGS. 4 and 5 can be performed by any of the devices/systems 114, 118, 200, and 300 described herein, and/or by various components thereof. In particular, any of the operations described with respect to the methods illustrated in FIGS. 4 and 5 may be performed by the DVT risk assessment system 124. Unless otherwise specified, and for ease of description, the method illustrated in FIG. 4 will be described below with reference to the environment 100 shown in FIG. 1, and the method illustrated in FIG. 5 will be described below with reference to the systems 200, 300 shown in FIGS. 2 and 3.

With reference to an example process 400 illustrated in FIG. 4, at operation 402, the DVT risk assessment system 124 may receive at least one ophthalmic image(s) of a patient, such as the patient 102. As described above with reference to FIG. 1, the ophthalmic image(s) may be captured by an optical imaging device 106 in a hospital setting (e.g., while the patient is hospitalized due to surgery or illness). The ophthalmic image(s) may include one or more images 108 of a retina and/or a fundus of at least one eye. As non-limiting examples, the ophthalmic images may comprise OCT or OCTA images, fluorescence angiograms, CFP images, and the like.

At an operation 404, the DVT risk assessment system 124 may include receiving electronic health record(s) of the patient. The electronic health record(s) (e.g., EMR data 112) may be accessed from an EMR system storing medical records of patients. The electronic health record(s) of the patient may include data indicating medical diagnoses and treatment of the patient during the current hospitalization, and a history of previous diagnoses and diagnostic tests, medications, or treatments received by the patient. In addition, the electronic health record(s) may indicate demographic information of the patient (e.g., age, sex, race, etc.), vital signs (e.g., blood pressure, blood oxygen level, heart rate, etc.), body mass index (BMI), lifestyle information (e.g., smoking or drug use status, physical activity level, diet, alcohol use, etc.), and the like, as measured during current and previous medical appointments.

At operation 406, the DVT risk assessment system 124 may include determining, by inputting features of the ophthalmic image(s) and the electronic health record(s) to one or more trained ML models, one or more potential diseases or disease risk(s) of the patient. In examples, the features of the ophthalmic images and the electronic health records may be determined by the image analysis system 120 and the EMR data extractor 122, as described with reference to FIG. 1. As described with reference to FIGS. 2 and 3, the ML models may comprise one or more ML models (e.g., CNN, RNN, transformer-based models, etc.) trained on large, anonymized training datasets containing retinal images, EMR data, and corresponding outcome of patients (e.g., whether they were diagnosed with DVT or PE) during their hospitalization. The one or more ML models may be trained to output a risk level associated with the patient for developing a DVT condition. In some examples, the ML models may accept, as inputs, retinal image features correlated with DVT and EMR data of the patient, and output a DVT/PE risk value. In some examples, the one or more ML models may comprise a decision tree, expert system, or a Bayesian belief network trained to compute a probability of developing DVT condition given the retinal image features and EMR data as input.

At operation 408, the DVT risk assessment system 124 may compare the DVT/PE risk value obtained at the operation 406 to a threshold. For example, if the DVT/PE risk value is higher than the threshold (Operation 408—Yes), the DVT risk assessment system 124 may generate, at an operation 410, a recommendation indicating a potential DVT condition requiring follow-up screening. In examples, the recommendation may be provided to the healthcare provider caring for the patient, and may be added to the electronic health record(s) of the patient.

Alternatively, if the confidence score is not higher than the threshold (Operation 408—No), the DVT risk assessment system 124 may generate, at an operation 412, an indication of normal status (e.g., no elevated risk of developing DVT/PE). In some examples, the indication of normal health status may not result in any action by the DVT risk assessment system 124 and/or the healthcare provider receiving the indication.

FIG. 5 illustrates an example process 500 for training a DVT risk assessment system, such as the DVT risk assessment system 124, to identify a risk of developing DVT/PE based on ophthalmic images and electronic health records of a patient. The process 500 may be performed by a computing device that includes at least one processor and memory. In some examples, the process 500 may be performed by a computing device that is different from the computing device 118 implementing the DVT risk assessment system 124, as described above with reference to FIG. 1.

At an operation 502, a training component of the DVT risk assessment system 124 may receive electronic health records and corresponding ophthalmic images of patients. In some examples, the electronic health records and the corresponding ophthalmic images may be received as a continuous process, as data instances (e.g., the training data 216) become available. As described, the electronic health records (e.g., from the EMR system 110) may include data indicating medical diagnoses and treatment of the patients during current hospitalization, and a history of diagnostic tests, medications, or treatments received by the patients. In addition, the electronic health record(s) may indicate demographic information of the patient, vital signs, lifestyle information, and the like, as measured over an extended period of time and/or during multiple medical appointments. The electronic health record(s) may also include physician's notes from the medical appointments, results of diagnostic tests performed on the patients, and treatment outcomes.

In examples, the ophthalmic images may include one or more of OCT images, slit lamp images, color fundus images, or retinal images captured by one or more medical imaging devices configured to obtain the ophthalmic images. In some examples, the ophthalmic images may also be stored in the EMR system in association with patients' electronic health records, and may include images captured at regular intervals during a hospital stay of the patient. In examples, the images may include retinal images illustrating blood clot-related conditions of a patient as well as images illustrating normal conditions. In examples, the electronic health records and the ophthalmic images of an individual patient may be identified as belonging to the same individual.

At an operation 504, the training component may receive DVT/PE outcomes of the patients. The DVT/PE outcomes may be based on results of follow-up screening, as described with reference to FIG. 2, which may include determinations by specialist medical professionals. In some examples, the DVT/PE outcome may be assigned as negative (e.g., no DVT/PE determined) after a patient is released from the hospital, or after a passage of a threshold amount of time with no blood clot-related complications reported for the patient.

At an operation 506, a training component may create the training dataset 216 for identifying DVT/PE risk in hospitalized patients. The training dataset may include features generated from the ophthalmic images and electronic health records e.g., as determined by the image analysis system 120 and the EMR data extractor 122. Each data instance (e.g., the training data 216) of the training dataset may correspond to an individual patient, and include the features, including standardized retinal images, of the individual and diagnoses indicating patient outcomes (e.g., whether the patient was diagnosed with DVT/PE) during a follow-up period of time. In some examples, the training dataset(s) may also include manual entries associated with one or more data instances, as provided by experts, indicating features (e.g., in the ophthalmic images) or diagnoses. In some examples, the training dataset may be created incrementally, as instances of training data (e.g., the training data 216) become available. For example, the training dataset(s) may be augmented to include a new batch of training data periodically, or when a threshold number of new instances of training data become available.

At an operation 508, the training component of the DVT risk assessment system 124 may include training, using the training dataset created at the operation 506, one or more ML models to determine a risk value for developing DVT/PE. For example, each training data instance may include a set of features as inputs, and a patient outcome for blood clot-related conditions (e.g., as indicated in the corresponding electronic health record) as a target output. In some examples, the one or more ML models may include one or more NNs (e.g., CNNs, RNNs, graph neural networks, transformers, etc.), and the training component may train the ML models based on optimizing parameter(s) of the models using techniques such as backpropagation. In some examples, the ML models may be decision trees, expert systems, or Bayesian belief networks, which may be trained by computing conditional probabilities of DVT condition given the set of features.

As discussed, the ML models trained by the training component may be used by the DVT risk assessment system 124 to generate a recommendation for screening of a patient determined to be at an elevated risk for developing DVT/PE condition. In examples, the process 500 may be repeated periodically e.g., in response to receipt of additional data and/or passage of time over a time threshold, to keep the ML models updated based on current data. For example, as discussed with reference to FIG. 2, the training dataset(s) may be augmented over time with the addition of more training data instances, such as the training data 216. The operation 508 may be repeated when a threshold number of training data instances have been added to the training dataset(s) after a previous training of the one or more ML models.

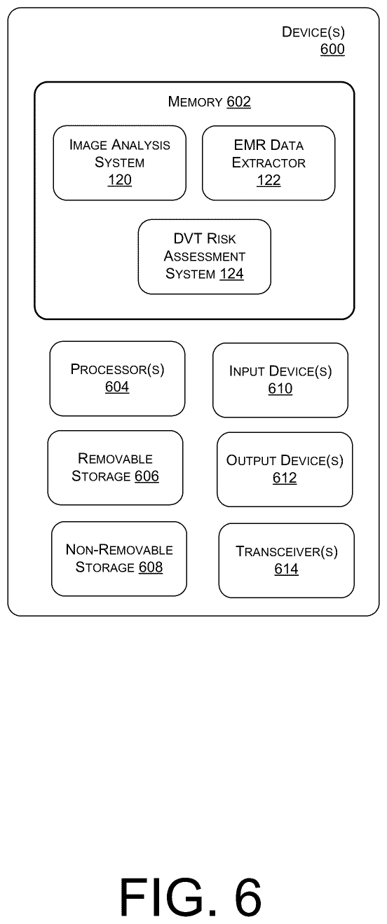

FIG. 6 illustrates at least one example device(s) 600 configured to enable and/or perform the some or all of the functionality discussed herein. Further, the device(s) 600 can be implemented as one or more server computers, a network element on a dedicated hardware, as a software instance running on a dedicated hardware, or as a virtualized function instantiated on an appropriate platform, such as a cloud infrastructure, and the like. It is to be understood in the context of this disclosure that the device(s) 600 can be implemented as a single device or as a plurality of devices with components and data distributed among them.

As illustrated, the device(s) 600, which may correspond to the computing device 118, may comprise a memory 602. The memory 602 can be used to store any number of functional components that are executable by the processor(s) 604. In examples, these functional components comprise instructions or programs that are executable by the processor(s) 604 and that, when executed, specifically configure the one or more processor(s) 604 to perform actions associated with providing a recommendation of screening for one or more the diseases. For example, the memory 602 may store one or more functional components, such as the image analysis system 120, the EMR data extractor 122, and the DVT risk assessment system 124, as illustrated in FIG. 1. The memory 602 may also include files and databases used by the one or more functional components. The memory 602 may include volatile and nonvolatile memory and/or removable and non-removable media implemented in any type of technology for storage of information, such as computer-readable instructions, data structures, program modules, or other data.

As described herein, the processor(s) 604, can be a single processing unit or a number of processing units, and can include single or multiple processing cores, comprising a Central Processing Unit (CPU), a Graphics Processing Unit (GPU), or both CPU and GPU, or other processing unit known in the art. For example, the processor(s) 604 can be one or more hardware processors and/or logic circuits of any suitable type specifically programmed or configured to execute the algorithms and processes described herein. The processor(s) 604 can be configured to fetch and execute computer-readable instructions stored in the memory 602, which can program the processor(s) 604 to perform the functions described herein. In some examples, a fast memory (e.g., SRAM) of the GPU may be used to implement I/O-aware attention mechanisms in a transformer-based architecture, which results in significant improvements in speed of training.

The device(s) 600 can also include additional data storage devices (removable and/or non-removable) such as, for example, magnetic disks, optical disks, or tape. Such additional storage is illustrated in FIG. 6 by removable storage 606 and non-removable storage 608. Tangible computer-readable media can include volatile and nonvolatile, removable and non-removable media implemented in any method or technology for storage of information, such as computer readable instructions, data structures, program modules, or other data. The memory 602, removable storage 606, and non-removable storage 608 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 600. Any such tangible computer-readable media can be part of the device(s) 600.

The device(s) 600 can also include input device(s) 610, such as a keypad, a cursor control, a touch-sensitive display, voice input device, etc., and output device(s) 612 such as a display, speakers, printers, etc. In some examples, the input device(s) 610 include a medical imaging device, such as the optical imaging device 106 described above with reference to FIG. 1. In particular implementations, a user can provide input to the device(s) 600 via a user interface associated with the input device(s) 610 and/or the output device(s) 612.

As illustrated in FIG. 6, the device(s) 600 can also include one or more wired or wireless transceiver(s) 614. For example, the transceiver(s) 614 can include a Network Interface Card (NIC), a network adapter, a LAN adapter, or a physical, virtual, or logical address to connect to the various base stations or networks (e.g., the network 116) contemplated herein, for example, or the various user devices and servers. To increase throughput when exchanging wireless data, the transceiver(s) 614 can utilize Multiple-Input/Multiple-Output (MIMO) technology. The transceiver(s) 614 can include any sort of wireless transceivers capable of engaging in wireless, Radio Frequency (RF) communication. The transceiver(s) 614 can also include other wireless modems, such as a modem for engaging in Wi-Fi, WiMAX, Bluetooth, or infrared communication.

Based at least on the description herein, it is understood that the DVT risk assessment system and devices and methods of the present disclosure may be used to assist in identifying an elevated risk of the patient developing a DVT/PE condition which may be potentially life-threatening, and recommending screening of the patients at risk. The DVT risk assessment system may be trained on a large training dataset of anonymized patient data, and provide a recommendation to the patient based on retinal images and EMR data of the patient, as collected during a hospital stay of the patient. The recommendation may allow for prevention of patient death by screening for development of DVT/PE condition in a hospitalized patient, to allow for early diagnosis and treatment before the condition becomes life-threatening.

The foregoing is merely illustrative of the principles of this disclosure and various modifications can be made by those skilled in the art without departing from the scope of this disclosure. The examples described above are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

What is claimed is:

1. A method, comprising:
receiving, by a processor, an image of a retina of an eye of a patient;
receiving, by the processor and from an electronic medical record (EMR) of the patient, patient data corresponding to the patient;
determining, by the processor, a feature in the image;
determining, by the processor and by inputting the feature and at least a portion of the patient data as input to a machine learning (ML) model, a risk level associated with a first condition;
determining, by the processor and based on the risk level being higher than a threshold, a recommendation for screening of the patient for the first condition; and
providing, by the processor and to an output device, an output indicating the recommendation.

2. The method of claim 1, wherein the feature comprises at least one of:
a cotton wool spot (CWS),
Roth spots,
a retinal vein occlusion (RVO),
a retinal hemorrhage, or
an emboli in a retinal blood vessel.

3. The method of claim 1, wherein the portion of the patient data is indicative of at least one of: venous statis risk factors, endothelial damage risk factors, or hypercoagulability risk factors.

4. The method of claim 3, wherein:
the venous statis risk factors comprise one or more of: sedentary lifestyle, hypertension, kidney failure, obesity, heart disease, or varicose veins;
the endothelial damage risk factors comprise one or more of: non-obstructive cardiovascular disease, hyperglycemia, hypertension, or hyperlipidemia; and
the hypercoagulability risk factors comprise one or more of: genetic factor, obesity, cancer diagnosis, medication, pregnancy, autoimmune disorder, smoking, infection, surgery, or immobilization.

5. The method of claim 1, wherein the recommendation is based at least in part on a length of stay in a hospital bed.

6. The method of claim 1, wherein the first condition comprises deep vein thrombosis (DVR) or pulmonary embolism (PE).

7. The method of claim 1, further comprising:
receiving, by the processor, follow-up information indicating whether the patient was diagnosed with the first condition;
augmenting, by the processor, a training dataset to include a data point comprising the follow-up information, the image, and at least the portion of the patient data; and
updating, by the processor, the ML model by re-training with the augmented training dataset.

8. The method of claim 7, wherein the training dataset includes anonymized patient data that:
are associated with a plurality of hospitalized patients,
include corresponding images of the retina of respective patients,
are extracted from an EMR system, and
include an indication of whether the respective patient was diagnosed with the first condition.

9. The method of claim 1, wherein the ML model is a first ML model, and determining the feature further comprises:
inputting, by the processor, the image to a second ML model; and
receiving, by the processor, and as output of the second ML model, an indication of the feature and a confidence level associated with the indication.

10. A system, comprising:
memory;
a processor; and
computer-executable instructions stored in the memory and executable by the processor to perform operations comprising:
receiving an image of a retina of an eye of a patient;
receiving, from an electronic medical record (EMR) of the patient, patient data corresponding to the patient;
determining a feature in the image;
determining, by inputting the feature and at least a portion of the patient data as input to a machine learning (ML) model, a risk level associated with a first condition;
determining, based on the risk level being higher than a threshold, a recommendation for screening of the patient for the first condition; and
providing, to the EMR of the patient, an output indicating the recommendation.

11. The system of claim 10, wherein the ML model is trained, based on a training dataset, to identify, based on the image and the patient data as inputs, the risk level associated with the first condition.

12. The system of claim 11, wherein the training dataset includes anonymized patient data and corresponding images of the retina associated with a plurality of patients, and an indication of whether one or more of the plurality of patients developed deep vein thrombosis.

13. The system of claim 10, the operations further comprising:
receiving follow-up information indicating whether the patient was diagnosed with the first condition;
augmenting a training dataset to include a data point comprising: the follow-up information, and the feature, and at least the portion of the patient data; and
updating the ML model by re-training with the augmented training dataset.

14. The system of claim 10, wherein the ML model is based on a transformer architecture.

15. The system of claim 10, wherein the first condition is deep vein thrombosis (DVT) or pulmonary embolism (PE) and the recommendation includes at least one of: imaging tests to confirm DVT, D-dimer test, or prescription of thrombolytic medication.

16. A non-transitory computer-readable storage medium storing processor-executable instructions that, when executed, cause one or more processors to:
receive, from an optical imaging device, an image of a retina of an eye of a patient;
access, from an electronic medical record (EMR) storage, EMR data of the patient;
determine a feature in the image;
determine, by inputting the feature and at least a portion of the EMR data as input to a machine learning (ML) model, a risk level associated with a first condition; and
determine, based on the risk level being higher than a threshold, a recommendation for screening of the patient based on the first condition.

17. The non-transitory computer-readable storage medium of claim 16, wherein the ML model is trained based on a training dataset comprising anonymized patient data and corresponding images of the retina associated with a plurality of patients, and an indication of whether the respective patient developed a blood clot-related condition.

18. The non-transitory computer-readable storage medium of claim 16, wherein the ML model is based at least in part on determining, in a training dataset, a correlation between the first condition and the feature or the EMR data.

19. The non-transitory computer-readable storage medium of claim 16, wherein:
the EMR data comprises at least one of: a blood pressure measurement of the patient, a length of hospital stay or surgical procedures performed during the hospital stay, and
the feature comprises at least one of: an emboli in a blood vessel of the retina, cotton wool spots (CWS), Roth spots, or a retinal vein occlusion (RVO).

20. The non-transitory computer-readable storage medium of claim 16, wherein the first condition comprises deep vein thrombosis (DVT) or pulmonary embolism (PE).
